(19) United States
(12) Patent Application Publication
(10) Pub. No.: US 2010/0278858 A1
(43) Pub. Date: Nov. 4, 2010

(54) PROCESS FOR PREPARING ATTENUATED VIRAL STRAINS

(75) Inventors: María Josefina Carlucci, Buenos Aires (AR); Elsa Beatriz Damonte, Buenos Aires (AR); Carlos Alberto Pujol, Buenos Aires (AR); Alberto Saúl Cerezo, Buenos Aires (AR); Marina Ciancia, Buenos Aires (AR)

Correspondence Address: THE NATH LAW GROUP, 112 South West Street, Alexandria, VA, 22314, US

(73) Assignee: INIS BIOTECH LLC, Milford, DE (US)

(21) Appl. No.: 12/743,952

(22) PCT Filed: Nov. 19, 2008
(86) PCT No.: PCT/IB08/54853

§ 371 Date: Jul. 16, 2010

(30) Foreign Application Priority Data
Nov. 20, 2007 (AR) P070105144

(51) Int. Cl. A61K 39/12; C12N 7/06; A61P 31/12; A61P 37/04

(52) U.S. Cl. 424/204.1; 435/238

(57) ABSTRACT

Provided is a process for preparing attenuated viral strains, comprising to contact, at least one sulphated polymer and a virus susceptible to the inhibition of the polymer, via successive passages of the virus with increasing polymeric concentrations, where the amenable virus is characterized by the method of reducing viral plates and where the strain resulting from the attenuated virus has stable phenotypic and genotypic characteristics, different from that of the virus strain in wild state that generated thereto. The process comprises to contact the sulphated polymer(s) with the virus susceptible to the inhibition of the polymer via about 15 or more successive passages with increasing concentrations of the sulphated polymer(s). According to the inventive process, the concentration of the at least one sulphated polymer in the first passage should be less than the IC₅₀ of the polymer for the amenable virus when it is found in wild state.

Provided is further use of the attenuated viral strains through the above mentioned process in the preparation of vaccines and pharmaceutical compositions.

The invention relates to a process for preparing attenuated viral strains, comprising to contact, at least one sulphated polymer and a virus susceptible to the inhibition of said polymer, via successive passages of the virus with increasing polymeric concentrations, wherein said amenable virus is characterized by the method of reducing viral plates and where the strain resulting from the attenuated virus has stable phenotypic and genotypic characteristics, different from that of the virus strain in wild state that generated thereto. The process comprises to contact the sulphated polymer(s) with the virus susceptible to the inhibition of said polymer via about 15 or more successive passages with increasing concentrations of said sulphated polymer(s). According to the inventive process, the concentration of said at least one sulphated polymer in the first passage should be less than the IC₅₀ of said polymer for the amenable virus when it is found in wild state.

Particularly, the invention refers to a process comprising to contact, via successive passages with increasing concentrations, at least one sulphated polymer and a virus susceptible to the inhibition of said polymer, to modify the viral behavior thereof as pathogenicity is referred to.

In one particular object of the invention, it also refers to the attenuated virus strains obtained by the process disclosed above and to the therapeutic or prophylactic compositions comprising thereof. Furthermore, the invention contemplates the vaccines comprising attenuated virus strains via the inventive process.

### BACKGROUND OF THE INVENTION

The sulphated polysaccharides are highly abundant and accessible compounds that may be isolated from various natural sources, and are known for their wide and variable physic-chemical properties which make them suitable for different applications in the medicine and pharmacology fields. They have shown to be useful due to their immuno-modulator and antitumoral activity, their interference in the clotting systems and in the inflammatory processes, in dermatology, in dietary programs and moreover by affecting the viral replication. Among the natural sources where they can be found are algae, constituting the cellular wall. Depending on the type of algae, those with similar structures to the glycosaminoglycans (GAGs) may be isolated, which may have a wide antiviral activity (Pujol C. A et al., 2007). Therefore, the effective action thereof for inhibiting a wide spectrum of enveloped virus may be mentioned such as the retrovirus: human immunodeficiency virus type 1 and 2 (HIV-1 and HIV-2), herpes virus: herpes virus types 1 and 2 (HSV-1 HSV-2), human cytomegalovirus (HCMV), pseudorabies virus; flavivirus: dengue virus type 2; smallpox virus: variola virus; hepadnavirus: hepatitis B virus (HBV); ortomixovirus: influenza A virus (inf A); paramixovirus: respiratory syncytial virus (RSV) and parainfluenza virus; rhabdovirus: vesicular stomatitis virus (VSV); arenavirus: Junin virus, Tacaribe virus y togavirus: Sindbis virus, Semliki Forest virus (table I) and against some naked virus, such us Encephalomyocarditis virus, Hepatitis A virus (Girond S et al., 1991) and papilloma-virus (HPV) (Buck C B et al., 2006), both DNA and RNA. For most of these viruses the initial bond of the virus to the cells would be mainly mediated by the virion interaction with a GAG type of the cellular surface known as heparan sulphate (Esko J D y Selleck S B, 2002).

Generally, it is known that the sulphated polysaccharides block the viral infection by chemically imitating the heparan sulphate, thus competing with the initial bond of the virus to the cellular surface.

TABLE I
Antiviral activity of sulphated polysaccharides
extracted from marine algae

| | | | |
|---|---|---|---|
| Algae | Compound | Virus | Reference |

Red Algae

| | | | |
|---|---|---|---|
| Schizymenia | Lambda- | HIV-1, AMV | Nakashima H et |
| pacifica | carrageenan | | al., 1987 |
| Schizymenia | Sulphated | HIV-1, HSV-1, | Bourgougnon N |
| dubyi | galactans with | HSV-2, VSV | et al., 1993 |
| | uronic acid | | |
| Nothogenia | (Xilo)mannans | HIV-1, HIV-2, | Kolender A et |
| fastigiata | | SIV, HSV-1, | al., 1997; |
| | | HSV-2, HCMV, | Damonte E et |
| | | Inf A, RSV, | al., 1994 |
| | | Junin, Tacaribe | |
| Aghardiella | Sulphated | HIV-1, HIV-2, | Witvrouw M et |
| tenera | Agarans | HSV-1, HSV-2, | al., 1994 |
| | | HCMV, VSV, Inf | |
| | | A, RSV, | |
| | | togavirus, | |
| | | parainfluenza | |
| | | virus, smallpox | |
| Digenea simples | sp sp non- | HIV | Sekine H et |
| | characterized | | al., 1995 |
| Fotogenia | Xilogalactans | HSV-1, HSV-2 | Damonte E et |
| fastigiata | | | al., 1996 |
| Pterocladiella | Sulphated | HSV-1, HSV-2, | Pujol C A et |
| capillacea | Agarans and | HCMV | al., 1996 |
| | hybrid DL- | | |
| | galactans | | |
| Gigartina | Lambda-, | HSV-1, HSV-2 | Carlucci M J et |
| skottsbergii | kappa/iota- and | | al., 1997, |
| | mu/un- | | 1999b |
| | carrageenans | | |
| Cryptopleura | Sulphated | HSV-1, HSV-2 | Carlucci M J et |
| ramosa | agarans | | al., 1997 |
| Stenogramme | Carrageenans | HSV-1, HSV-2 | Cáceres P J et |
| interrupta | | | al., 2000 |
| Asparagopsis | Sulphated | HIV | Haslin C et |
| armata | agarans | | al., 2001 |
| Bostrychia | Sulphated | HSV-1, HSV-2 | Duarte M E et |
| montagnei | agarans | | al., 2001 |
| Gymnogongrus | Hybrid DL- | HSV-2, virus | Pujol C A et |
| torulosus | galactans | dengue 2 | al., 2002 |
| Gracilaria | Sulphated | HSV-1, HSV-2 | Mazumder S et |
| corticata | agarans | | al., 2002 |

Brown algae

| | | | |
|---|---|---|---|
| Pelvetia | Fucans | HBV | Venkateswaran |
| fastigiata | | | P S et al., 1989 |
| Fucus | Fucans | HIV-1 | Beress A et |
| vesiculosus | | | al., 1993 |
| Sargassum | Fucans | HSV-1, HCMV | Hoshino T et |
| horneri | | HIV-1 | al., 1998 |
| Leathessia | Fucans | HSV-1, HSV-2 | Feldman S C et |
| difformis | | | al., 1999 |
| Adenocystis | Fucans | HSV-1, HSV-2 | Ponce N M A et |
| utricularis | | | al., 2003 |

Microalga

| | | | |
|---|---|---|---|
| Cochlodinium | Extract | HIV-1, RSV, Inf | Hasui M et al., |
| polykrikoides | | A, Inf B | 1995 |
| Porphyridium sp | Extract | HSV-1, HSV-2 | Huheihel M et |
| | | | al., 2002 |

Green alga

| | | | |
|---|---|---|---|
| Monostroma | Sulphated | HSV-1, HCMV, | Lee J B et al., |
| latissinum | Rhamnans | HIV-1 | 1999 |

GAGs are long chains of unbranched polysaccharides, formed by the successive repetition of the disaccharides unit, which may be sulphated. GAGs may be divided in two categories: glucosaminoglicans, such as heparan sulphate, and galactosaminoglicans as the chondrointin sulphate. As it can be seen from its name, a defined difference is the initial introduction of saccharides units N-acetil-glucosamine or N-acetil-galactosamine respectively. Another important difference is that the glucosaminoglicans are linked in a series of saccharides bonds 1,4, while the galactosaminoglicans are linked in an alternate series 1,3 and 1,4. GAGs are mainly located on the cellular surface and in most of the intercellular matrix of the mesodermal tissues as it is shown in Table 2 (connective, cartilage, muscle and bone) and, frequently, they are linked to a protein core, thus forming the so-called proteoglicans. GAGs are molecules negatively charged that may have physiologic significance, as for example, the hialuronic acid, dermatan sulphate, chondroitin sulphate, heparin, heparan sulphate and keratan sulphate.

TABLE II

| GAG | Location | Comments |
|---|---|---|
| Hialuronates | Synovial fluid, | Long polymers |
| | vitreous humor, | (containing no |
| | extracellular matrix | sulphates), shock- |
| | with loss of | absorbing. |
| | connective tissue | |
| | (vasculogenesis). | |
| Chondrointin | cartilages, bone and | More abundant GAGs. |
| sulphate | cardiac valves. | |
| Heparan sulphate | Basal Membrane and | |
| | components of the | |
| | cellular surface. | |
| Heparine | Components of the | More sulphated than |
| | intracellular | the Heparan |
| | granules of the | sulphate. |
| | mastocytes, coating | |
| | of the lung | |
| | arteries, liver and | |
| | skin. | |
| Dermatan sulphate | Skin, cardiac valves | Long polymers (no |
| | and blood vessels. | sulphates), shock- |
| | | absorbing. |
| keratan sulphate | Cornea, bone and | More abundant GAGs. |
| | cartilage. | |

The polyanionic substances tested as antiviral may be classified according to the anion present in the molecule. The polysulphonates represent the largest class of polianions characterized as antiviral, among which a wide spectrum of sulphated polysaccharides and polyvinyl alcohol derivatives are included, polyacetals, naphthalenes, polystyrenes and other polymers. In this field, various studies have been carried out about the antiviral properties of the sulphated polysaccharides isolated from marine algae or the synthetic analogues thereof, as well as those isolated from other natural sources, as for example, the higher plants, the marine invertebrates and the cyanobacteria (Damonte E B et al., 2004).

Red algae contain large quantities of polysaccharides in the cellular wall thereof, most of which are sulphated galactans. These galactans are generally constituted by alternately repeated units of bonds 1,3-α-galactopyranose and 1,4-β-D-galactopyranose and defer in the level and pattern of sulphation, in the substitution by methoxy and/or piruvate groups and other sugars such as mannose and xylose. They also defer in the 3,6-anhidrogalactose content and the 1-3-α-galactopyranose residues configuration.

Among these galactans, the carrageenans may be mentioned, which have similar structures to the pattern observed in the galactosaminoglicans. These compounds are largely used in the food industry and in the biotechnology industry as gelling agent and thickeners. They comprise a wide group of structures and may be divided in two families: the κ-family, defined by the presence of a sulphated C4 group in the unit β-D, and formed by carrageenans-κ/τ and the carrageenans-μ/ν, and the λ-family, characterized by a sulphate-C2 group and constituted by all the varieties of λ structures (Painter T J, 1983). The λ- and τ-carrageenan types are more strongly sulphated than the most of the heparan sulphate derived from tissues (Esko J D and Selleck, 2002). In general, this type of carrageenans exhibits a viral inhibitory potential a little greater than the κ-carrageenans.

Recently, it has been reported that in the course of an inflammation, an infection or tissue damage, the proteoglican heparan sulphate is cleaved causing fragments of soluble heparan sulphate (Ihrcke N S et al., 1998). On the other hand, in healthy tissues, no significant fractions of soluble heparan sulphate are found, though they can be found in the fluids of damaged tissues—at concentrations within the required ranges to stimulate dendritic cells (Kainulainen V H et al., 1998)—and in the infected individuals' urine (Oragui, E et al., 2000).

Accordingly, a sulphated polymer having a chemical structure similar to some GAG (for example, the cellular heparan sulphate), which has an inhibitory activity for a determined virus (for example, being an inhibitor of the herpes simplex virus) and whose mechanism of action affects certain stage of the viral cycle, would generate, under selection pressure, viral variants resistant to said compound. At the same time, the modified virus-compound bond sites might interfere with other functions as antigenic determinants or virulence expression sites.

As from the above discussion, but without being bond to the a particular hypothesis, the present inventors believe that said viral variants might spontaneously occur as a consequence of the similar structure of the carrageenans with the cellular heparan sulphate, causing phenotypic and genotypic modifications, and affecting the viral envelope as the (g) gB, gC ó gD glycoproteins.

On the other hand, the HSV has developed multiple immune evasion strategies to respond to the host attacks during the infection and the reactivation. Some of the mechanisms comprise:

1) viral escape, due to alterations in the viral envelope or for the reduction of the viral expression during the latency phase.

2) viral resistance such as the sequential induction of the pro- and anti-apoptotic effects of the defense cells.

3) viral counterattack inhibiting the dendritic cells ageing (Novak N y Peng W M, 2005).

Some determinants for the immune evasion may be found in the HSV-1 surface glycoproteins, which are expressed on the viral envelope and in the surfaces of the infected cells. Thus, the gB interacts with the HLA-DR and HLA-DM polypeptides, by reducing the expression of the invariable chain and interrupting the presentation of the MHC II antigen (Neumann J et al., 2003). The gC intervenes avoiding the neutralization mediated by the complement (Friedman H M et al., 2000) and, also, would be involved in the adding of the monocytes to the endothelial cells (Larcher Cl et al., 2001). On the other hand, gD induces the activation of the κB (NF-κB) nuclear factor and the protection thereof against the apoptosis in the early phase of the infection, assuring a sufficient viral replication (Novak N and Peng W. M, 2005). Therefore, alterations in these glycoproteins generated by the successive interactions of endogenous soluble or exogenous polysaccharides (for example, microbicides), might modify the normal behavior thereof, providing asymptomatic or subclinical infections. Particularly, variants obtained by the inventive process, would be involved in the above mentioned point 1, as the alterations in the glycoproteins are directly shown by the resistance generated to the original drug or similar compounds, a different cytopathic action with respect to the pattern strain and greater in vitro spreading rate. Moreover, the genetic modifications of the viral thymidine kinase (TK) would result in a decrease or an annulment of the viral reactivation since the latency (Efstathiou S et al., 1989, Evans J, et al., 1998).

Carrageenans, sulphated polysaccharides of various structural types, isolated from the red alga Gigartina skottsbergii, have been identified as potent and selective inhibitors of the HSV-1 and the HSV-2. The study of the mechanism of action of the carrageenans over the HSV replication suggests that they would mainly affect the adsorption, initial stage of the viral cycle, involving surface glycoproteins (Carlucci M J et al., 1999a; Carlucci M J et al., 1999b). On the other hand, it is known that the frequent use of antivirals generates resistance, which was confirmed in vitro with HSV-1 and successive passages to increasing concentrations of carrageenan μ-ν 1C3 (Carlucci M J et al., 2002), obtaining variants with different cytopathic characteristics, drug resistance and virulence.

Therefore, due to the similarity among the heteropolysaccharides that constitute the cellular GAGs and the sulphated structures of the natural or synthetic polymers, the present inventors have proposed that by successive passages with increasing concentrations of the above mentioned compounds, which antiviral action would be involved in the first stages of the replication cycle, it would be possible to attenuate the pathogenic action of certain amenable viruses, and thus being possible to use them for therapeutic and prophylactic purposes.

Some processes used for the production of attenuated viral strains of the previous art are described below:

1. Serial passages of the wild virus in another different host from that in which the disease is caused. Therefore, the variant that generally loses the virulence for the primary host is selected. A hazardous method which generally causes mixed populations from which the attenuated viral strain is cloned to be characterized.

2. Mutating a wild virus stock and analyzing the surviving viral particles that may have lost the virulence by random mutation in a gene responsible thereof. Boring and meticulous method, which not always provides the expected results.

3. Random isolating any infected host within the ecosystem, an attenuated viral strain. Example, the measles vaccine strain.

4. Virus which genome has been totally sequenced, it is possible to introduce nucleotides in specific sites (site-directed mutagenesis) or to remove them in order to alter the genic sequence responsible for the virulence. It is only useful in cases where the virulence depends on the expression of a unique gene.

However, the processes used for the production of attenuated viral strains of the previous art, show, among others, the following disadvantages:

1. Possible reversion to the virulence of the strain used to vaccinate, once it is introduced in a subject and is removed by the subject to the environment, as is the case of the vaccine against the poliomyelitis virus.

2. Unseen contamination of the vaccine seed with other virus, as for example, the first batches of the poliomielytis attenuated vaccine with SV40 virus coming from the cellular substrate, a primary line of monkey kidney. Presently, the use of human embryonic lines is recommended for vaccines substrates, to avoid risks.

3. It is not advisable for pregnant women and immuno-compromised people.

However, in spite of all the inconveniences, the attenuated virus vaccines are those which have allowed stopping some of the most important virosis for men, such as smallpox, poliomyelitis, measles and rubella.

In fact, vaccines are based on attenuated live virus, and have, among others, the following advantages:

1.—They are good immunogen.

2.—They induce a large and intense immunity. This is due to the fact that the virus replicates in the organism, being the multiplication thereof limited, without reaching important organs, since the immune system stops the infection giving place to an immunological memory similar to the natural infection.

3.—In general, for an efficient immunization, a unique vaccine dose may be enough. The maintenance of the immunitary protecting level is made through the subsequent natural reinfections or for the administration of a recall dose.

4.—They are administered by inoculation, respiratory and ingestion way. These ways confer immunity both humoral and local, avoiding infection at the entrance door of the microorganism and the subsequent spreading thereof.

Therefore, the present inventors have developed a new and advantageous process for producing the attenuated viral strains, which may satisfactorily resolve many of the disadvantages of the previous art processes.

### BRIEF DESCRIPTION OF THE INVENTION

The present invention refers to a process for making the attenuated viral strains, comprising to contact, at least one sulphated polymer and a virus susceptible to said polymer inhibition, by successive passages of the virus with increasing concentrations of the polymer, where said amenable virus is characterized by the method of reducing the viral plates and where the attenuated virus strain has stable phenotypical and genotypical characteristics, different to that of the wild type virus strain that generated thereto.

The invention also refers to the use of attenuated virus strain by the process hereby claimed, where said virus strain is used in the making of vaccines or pharmaceutical compositions with therapeutic and prophylactic activity.

### DETAILED DESCRIPTION OF THE INVENTION

The invention refers to a process for making the attenuated viral strain, comprising to contact, al least one sulphated polymer and a virus susceptible to said polymer inhibition, through successive passages of the virus with polymer increasing concentrations, where said amenable virus is characterized by the method of reducing viral plates and where the attenuated virus strain has stable phenotypical and genotypical characteristics, different to that of the wild type virus strain that generated thereto. The process comprises to contact the sulphated polymer(s) with the virus susceptible to the inhibition of said polymer by about 15 or more successive passages with increasing concentrations of said sulphated polymer(s). According to the inventive process, the concentration of at least one sulphated polymer in the first passage should be less than the IC₅₀ of said amenable virus when it is in wild type state.

Particularly, the invention refers to a process comprising to contact, through successive passages with increasing concentrations, at least one sulphated polymer and a virus susceptible to the inhibition of said polymer, to modify the viral behavior thereof as pathogenicity is referred to.

In a specific objective of the invention, it also refers to the attenuated virus strains produced by the above disclosed process, the use thereof and the therapeutic and prophylactic compositions comprising thereof. Moreover, the invention contemplates the vaccines comprising at least an attenuated virus strain by the inventive process.

According to the present invention, an attenuated viral strain means a live viral strain that cannot cause a disease, though it infects cells and replicates within the organism. So, once in contact with them, the immune system may be prepared to protect the organism from the reinfection with pathogenic strains (Basualdo J A et al., 2006).

According to the present invention, the attenuated viral strains have the viral structure thereof altered. Particularly, the attenuated virus strains produced by the inventive process have the envelope thereof altered.

Thus, it is also a particular object of the invention a virus strain produced by a process comprising to contact, at least one sulphated polymer and a virus susceptible to the inhibition of said polymer, through successive passages of the virus with increasing concentrations of the polymer, where said amenable virus is characterized by the method of reducing viral plates and where the strain resulting from the attenuated virus has stable phenotypical and genotypical characteristics, different to that of the wild type virus strain that generated thereto. Said attenuated virus strain has the viral structure modified and is resistant to the sulphated polysaccharides of similar structure to that used in said process. Moreover, it has an IC₅₀ that is about 4 times greater than the IC₅₀ of the wild type virus. According to the present invention, the attenuated virus strains are resistant to drugs having a different mechanism of antiviral action than that of the sulphated polysaccharide which have been contacted with, (for example: Brivudine, Heparine, Aciclovir and Foscarnet), wherein the resistance is not a determining factor of attenuation.

According to the present invention, a method of reducing the viral plates comprises the following process: Vero cells monolayers developed in 24 wells microplates are infected with 50 UFP/well of virus in the absence or presence of different compounds concentrations. Each dilution is tested by duplicate. After 1 hour adsorption at 37° C., the inoculum is removed and covered by plating medium (culture media with methylcellulose at a final concentration of 0.7%). Inoculation is continued till the viral plates' appearance, which are then counted after fitting and dying the cells with violet crystal. Moreover, according to the present invention, 50% inhibitory concentration (IC₅₀) means the compound concentration required for reducing about 50% the number of viral plates.

Furthermore, as it is used in the present document, wild type virus means the viral strain used as a reference, that comes from infected patients and have scarce passages in cellular cultures keeping proper characteristics.

By the inventive process attenuated strains both of RNA and DNA virus, either enveloped or naked, may be produced. Particularly, the process is specifically useful for the making of attenuated viral strains of the Herpes virus. Preferably, said Herpes virus is the Herpes simplex virus type 1 or the Herpes simplex virus type 2.

Preferably, the sulphated polymer is selected from the natural sulphated polymers and the synthetic sulphated polymers. In particular, the sulphated polymer is a glycosaminoglican or a sulphated polymer with similar structure to a glycosaminoglican. More preferably still, the sulphated polymer is selected from the carrageenans. Moreover, among the synthetic sulphated polymers, the synthetic naphthalene sulphated polymers are preferred.

In a particular object of the invention, a process for preparing attenuated viral strains, comprising to contact, at least one sulphated polymer and a virus susceptible to the inhibition of said polymer, via successive passages of the virus with increasing polymeric concentrations, where said amenable virus is selected by the method of reducing viral plates and where the strain resulting from the attenuated virus has stable phenotypic and genotypic characteristics, different from that of the virus strain in wild state that generated thereto. The process comprises to contact at least one sulphated polymer, preferably a sulphated polymer and more preferably still a carrageenan, with the virus susceptible to the inhibition of said polymer via about 15 or more successive passages with increasing concentrations of said sulphated polymer(s). According to the inventive process, the concentration of said at least one sulphated polymer in the first passage should be less than the IC₅₀ of said polymer for the amenable virus when it is found in wild state.

In particular, the inventive process might also comprise one or more of the following stages:

1. Determining if the compound to be used has killing activity against the virus under study and defining the 50% viral concentration thereof (CV₅₀).

2. Comparing the 50% antiviral and killing inhibitory concentrations

3. If both concentrations are similar (IC₅₀ y CV₅₀), considerably extending the time for the variants selection, starting from the serial passages with an initial sub-dose of the compound about 5 to 10 times lower than the IC₅₀ and reasonably increasing the following passage concentration in once to twice the previous one.

4. If the compound has no killing effect, employing an initial concentration of about twice to four times lower than the corresponding IC₅₀ thereof, to avoid a rough decreasing of the viral title and a gradual adjustment of the viral particles to the medium

5. Incubating the cellular monolayers till a cytopathic manifestation between 70-90% is observed.

6. Titling each passage, carrying out passage amplification to the passage that has decreased about 2 log the original viral title thereof.

7. Carrying out a viral cloning between passages 13-15 in order to analyze the resistance characteristics of the selected clones to different drugs, preferably selecting those who show certain phenotypical change, as for example plate size or syncytium formation.

The inventive process presents, among others, the following advantages:

1. Serial passages may be made in different monolayers of cellular lines, including diploid human cells, avoiding the contamination with adventive virus, related to the cellular substrate.

2. Sulphated polymers are used as agents for the selection of attenuated viral mutants, which are more abundant in nature, they are easily collectable and obtainable, stable, cheap, innocuous, of variable structure and easily chemically modifiable, allowing the incorporation of a large number of sulphated groups.

3. Same methodology may be used with different types of natural or synthetic sulphated polymers (as for example PRO2000).

4. The process of preparing the mutant strains of the invention does not require sophisticated equipments or complicated or expensive methodologies.

5. Many of the sulphated polysaccharides of a structure similar to GAGs that may be used in the process of the invention, are largely spread in the organism and keep a constant selection pressure, due to the molecules circulation as, for example, heparine or heparan sulphate, originated by the cellular exchanges, infections or inflammatory processes, thus controlling the risk of reversing to the virulent form of the virus.

6. The sulphated polysaccharides modify not only the viral structure, but also other genomic sites of expression of virulence as the thymidine kinase gene. This reinforces the possibility of having an attenuated viral strain with great stability especially in the case of those viruses which virulence does not depend on the expression of a unique gene.

7. It allows preparing a vaccine formed by mixed serotypes, made by the same process and agent.

The formulation of a vaccine or pharmaceutical composition with therapeutic or prophylactic activity, comprising attenuated live virus according to the process of the present invention, may be determined by the expert in the art through known techniques and may be found in the available bibliography.

### PREPARATIVE EXAMPLES

### Selection of Viral Variants with SAMMA and PRO2000 Through Successive Passages

For the selection of viral variants in presence of SAMMA (non-sulphated polymer from the condensation of mandelic acid) and PRO2000 (naphthalene sulphated polymer), CaSki cells were infected (human cervical carcinoma cells) with HSV-2 (G) with an infection multiplicity of 0.1 UFP/cell in the presence of an initial concentration of 2.5 μg/ml SAMMA (IC₅₀SAMMA: 4.2 μg/ml) and of 0.25 μg/ml PRO2000 (IC₅₀ PRO2000: 0.2 μg/ml). In case that the compounds employed would have killing action, it was considered convenient to start the passages with subdoses, to avoid the major inhibition of the virus. Compounds were present during the viral adsorption (1h, 37° C.) and after the infection. When a cytopathic action (ACP) between 80-90%, during a period among 24 to 72 hours p.i., was observed, cells were lysed with freeze-thawing cycles. In order to remove cellular moieties, the cellular lisate was centrifuged during 10 min at 1000 rpm, keeping the supernatant at −70° C. The supernatant was used to infect a new monolayer in the presence of SAMMA or PRO2000. When a massive cytopathic effect was observed at a short time, indicating a high viral title, concentration of each compound was duplicated. The title and the IC₅₀ of each viral passage was determined by viral plates reduction test (or after 2-3 passages) against SAMMA or PRO20000.

By the end of a year, the corresponding virus to passages 13 and 16 in the presence of PRO2000 and SAMMA, respectively, were cloned in monolayers of Vero cells developed in 6 wells plates. The cellular monolayers were infected with a limited dilution of virus and were coated with maintenance media (MM) containing agarose 0.6%. After 2 days incubation, cells were dyed with neutral red and clones were stippled. For clone amplification, Vero cells with each selected clone were infected; after 1 hour adsorption at 37° C., the innoculum was discarded and was covered with MM, subjected to incubation 24-48 h at 37° C. till ACP development was observed. Clones were titled and in vitro susceptibility of antiviral compounds was subsequently shown.

Simultaneously, serial passages of HSV-2 (G) were made as a control in CaSki cells without SAMMA or PRO2000. In the same way, variants with 1C₃ were obtained as previously described (Carlucci M J et al, 2002).

Stability of viral strains was confirmed by carrying out 5-10 passages in Vero cells in the absence of compounds.

The 1C3-syn13-8 and 1C3-syn14-1 variants already studied showed variability in the cytopathic action depending on the cellular type tested, generating similar rounds to the wild strain or syncytio (Table III).

TABLE III
Syncytial phenotype and cellular type

| | | | | | | | Murine |
|---|---|---|---|---|---|---|---|
| | | | | | | Murine | Macro- |
| Virus | Vero | CV1 | PH | CasKi | Hep-2 | Astrocites | phages |
| HSV-1 | No | No | No | No | No | No | No |
| (F) | Syn | Syn | Syn | Syn | Syn | Syn | Syn |
| Syn- | Syn | Syn | No | Syn | No | No | — |
| 13-8 | | | Syn | | Syn | Syn | |
| Syn- | Syn | Syn | No | Syn | No | No | Syn |
| 14-1 | | | Syn | | Syn | Syn | |

Vero: Cercopithecus aethiops African green monkey kidney
CV-1: derived from green monkey kidney.
HEp-2: epithelial carcinoma of human larynx
PH: human prepuce.
CasKi: human cervical carcinoma.
Syn: syncytial.

Noticeable in vivo pathogenicity changes were also shown depending on the inoculation via (Carlucci M J et al., 2002). Thus, by intravaginal via, 1C3-syn13-8 and 1C3-syn14-1 generated a mortality rate of 60% and 0% respectively, in comparison with 100% of control (HSV-1 strain F). With the same processes but with HSV type 2 strain G and the PRO2000 sulphated polymer, strains of similar characteristics were isolated (PRO2000-syn 13-9 and PRO2000-syn 13-1) at Mount Sinai School of Medicine, New York, N.Y. (Carlucci et al., non published results). This allows stating that this class of compounds, repeatedly used, may modify the viral behavior as pathogenicity is referred to.

Moreover, by the same methodology, variants with other non-sulphated compound were obtained: SAMMA. The strains so generated were not synsytial and the SAMMA-15-3 and SAMMA-15-7 analyzed showed resistance to the same drug, to the heparine and the carrageenan IC₃ in the order of 2.6 to 6.7 times with respect to the control virus.

In vivo, SAMMA-15-3 and SAMMA-15-7, showed a rare difference of pathogenicity and mortality with reference to the wild strain HSV-2 (G). However both the viral variants obtained with PRO2000 and those obtained with IC₃, showed syncytial characteristics, decreasing virulence and mortality in an 80% with the PRO2000-13-9 strain (surviving would be obtained if the selection pressure with 2-3 passages more was continued) and 100% with 1C3-syn-14-1 with respect to control.

TABLE IV
Susceptibility of viral variants to anionic polymers

| | IC50^{a} (RR^{b}) | | | |
|---|---|---|---|---|
| | Carrageenan | | | |
| Virus | 1C3 | Heparine | SAMMA | PRO2000 |
| HSV-1 (F) | 1.4 | 1.4 | 4.0 | 0.3 |
| 1C3-syn-13-8 | 1.6 (1.1) | 1.2 (0.9) | 10.0 (2.5) | <0.15 (0.5) |
| 1C3-syn-14-1 | 6.3 (4.5) | 4.1 (2.9) | 2.1 (0.7) | <0.15 (0.5) |
| HSV-2 (G) | 4.5 | 1.8 | 4.2 | 0.2 |
| SAMMA-15-3 | 14.0 (3.1) | 8.4 (4.6) | 17.9 (4.3) | 0.1 (0.5) |
| SAMMA-15-7 | 11.7 (2.6) | 12.0 (6.7) | 19.0 (4.5) | 0.1 (0.5) |
| Pro2000- | — | 7.8 (4.3) | — | 0.3 (1.5) |
| syn13-1 | | | | |
| Pro2000- | >20 (4.4) | 19.5 (10.8) | — | 0.2 (1.0) |
| syn13-9 | | | | |
| Pro2000-13-5 | — | 2.2 (1.2) | — | 0.2 (1.0) |
| Pro2000-13-10 | — | 2.6 (1.4) | — | 0.2 (1.0) |

IC50^{a} (50% inhibitory concentration) is the drug concentration in μg/ml required to decrease the number of plates in 50%.
RR^{b} (relative resistance) is the relation between the IC50 of each viral variant and the IC50 for the HSV-1 (F) ó HSV-2 (G) control strain.

As it can be seen in Tables IV and V, from the HSV-1 (F) and HSV-2(G) wild strains, with no syn characteristics, susceptible to all the tested compounds and producing a 93-100% mortality of the infected animals, variants with the following characteristics were obtained:

a. Pathogenic variant with 1C3-syn-13-8 synsytial phenotype, sensitive to 1C3, heparine and Pro2000.

b. Non-pathogenic variant with 1C3-syn-14-1 synsytial phenotype resistant to 1C3, heparine and sensitive to Pro2000.

c. Variant of low pathogenicity with Pro2000-syn-13-9 synsytial, resistant to 1C3 and to heparine, sensitive to Pro2000.

d. Non synsytial pathogenic variant SAMMA-15-3, resistant to 1C3, heparine, SAMMA and sensitive to PRO2K.

The viral variants obtained by selection pressure with different polymers would have the viral structure altered (probably the envelope thereof) involving stable phenotypical and genotypical changes, which would modify the susceptibility to the drug that generated thereto as well as other related compounds and the pathogenicity thereof.

Independently of the viral serotype (HSV-1 ó HSV-2) and of the sulphated polymer employed to generate said variants (1C3 and PRO2000), avirulent mutants could be obtained as in the cases of 1C3-syn-14-1 and PRO2000-syn-13-9.

It is worthwhile wondering if these statements could be extrapoled to other enveloped virus following the same methodology and if the asymptomatic variants are more susceptible to be selected and/or generated with sulphated polymers than those lacking of sulphation with SAMMA.

On the other hand, according to the different above listed variants, we could infer that the synsytial phenotype would not be related to the pathogenicity observed in vivo as 1C3-syn-13-8 shows. Besides it could be said that the resistance to the original drug is not the main determinant to define an attenuated mutant as PRO2000-syn-13-9 shows.

TABLE V
Evaluation of the in vivo viral virulence
in a vaginal infection model.

| | *Title (in | Mortality | | |
|---|---|---|---|---|
| | vaginal washings) | | | Average |
| Virus | (UFP/ml) | N°/total | % | Day |
| HSV-2 (G) | 3.2 × 10⁴ | 10/10 | 100 | 8.6 ± 1.3 |
| HSV-1 (F) | 1.4 × 10⁴ | 14/15 | 93.3 | 7.6 ± 0.9 |
| Pro2000-syn13-9 | 1.7 × 10⁴ | 2/10 | 20 | 8.0 ± 0 |
| 1C3-syn13-8 | 1.6 × 10⁴ | 6/10 | 60 | 7.2 ± 1.5 |
| 1C3-syn14-1 | 3.7 × 10⁴ | 0/15 | surviving | Surviving |
| Samma-15-3 | 6.1 × 10⁴ | 7/10 | 70 | 9.2 ± 1.5 |
| Inactivated HSV- | — | 0/5 | Surviving | Surviving |
| 2 (G) | | | | |
| PBS | — | 0/3 | Surviving | Surviving |

The virulence of the strains used (HSV-1 (F), HSV-2 (G)) as well as the respective variants thereof, showed different signs and symptoms of the infection to be inoculated via intravaginal (Table 3 and 4). Thus, mortality for HSV-2 (G) was classified as: 1) non-apparent infection, 2) vaginal redness, 3) moderate erythema or vaginal inflammation and surrounding tissue, 4) severe erythema or perivaginal inflammation 5) ulceration or severe inflammation, erythema or alopecia, 6) neurological disease. For HSV-1 (F): 1) non-apparent infection, 2) vaginal redness, 3) colonic constipation and moderate perineal inflammation, 4) stomach distention and severe urinary sphincter and anal obstruction, 5) neurological disease with abdominal necrosis.

Animals that showed level 4 of said classification for both viruses, were sacrificed according to manipulation processes and animal care in accordance with national and international laws and policies (Regulation for care and use of test animals, Facultad de Ciencias Exactas y Naturales, Buenos Aires, Argentina, approved by CD 140/00, y and the Helth Department and Human Service, Public Health Service, NIH, 2002. Assurance Identification #A5523-01).

TABLE VI
Comparative chart of the polymers used in the variants study

| | | | | | Syn (%) | |
|---|---|---|---|---|---|---|
| | | | | Virus | (Passage | Mortality* |
| Compound | Polymer | Sulphated | Origin | employed | N°) | (%) |
| 1C₃ | Galactan | SI | Natural | HSV-1 F | 90 (14) | Surviving |
| PRO2000 | Naphthalene | SI | Synthetic | HSV-2 G | 20 (13) | 20 |
| SAMMA | Mandelic acid | NO | Synthetic | HSV-2 G | 0 (15) | 70 |

*Mortality referred to infection via intravaginal.

Furthermore, results disclosed that intravaginal infections in a murine model with the HSV-1 (F) strain and the 1C3-syn-14-1 viral variant thereof, produce a minimum or null response to pro-inflammatory cytokines, IFNγ, and interleukines such as IL-6 and TNF-α (Table VII). Infection passes asymptomatically with a 100% surviving, though in vaginal washings the same quantity of infective virus as in the control is recovered. While for the HSV-2 (G) and the PRO2000-syn13-9 viral variant thereof, the levels of the altered cytokines were different in comparison with HSV-1, such as IL-1β and IFN-γ, having relation with the animals' symptomathology differences. In both cases the viral variants both for HSV-1 and HSV-2 showed a significant reduction of the virulence after the selection pressure made with the sulphated polymers PRO2000 and 1C3. As in any viral infection, the immune system response is important as it induces the production of antiviral factors (such as interferons), which prevent the replication and consequently the viral spreading. The inflammatory cytokines also play an important role (such as IL-6 and TNF-α), which recruit and activate effector cells, though sometimes may make the condition worse and they are frequently responsible for the symptoms shown in the disease (fever, myalgias, pain related with local inflammation). As it occurs with the bacterial sepsis, the inflammatory response via cytokines may, in some cases, lead to morbility or mortality, for example in the case of the herpetic encephalitis, as a consequence of the TLR-2 activation resulting in brain inflammation and death (Kurt-Jones E et al., 2004).

TABLE VII
Determination of cytokines and chemokines in murine vaginal washings

| | Inactivated | HSV-1 | 1C3syn | HSV-2 | PRO2K-syn |
|---|---|---|---|---|---|
| Cytokine | HSV-2 (G) | (F) | 14-1 | (G) | 13-9 |
| TNFα* | 0.86 ± 1.0 | 37.10 ± 2.0 | 7.50 ± 0.4 | 18.05 ± 4.9 | 7.50 ± 0.4 |
| IL-1β* | 476.2 ± 0 | 1268.53 ± 116.4 | 1060.16 ± 4.8 | 276.6 ± 32.4 | 925.2 ± 23.9 |
| IL-6* | 9.05 ± 0 | 4040.3 ± 69.2 | 512.74 ± 24.8 | 381.5 ± 37.8 | 408.3 ± 4.7 |
| IL-2• | 0.58 ± 0 | 1.75 ± 1.6 | 0.58 ± 0 | 0.58 ± 0 | 0.58 ± 0 |
| IL-4• | — | 4.95 ± 1.0 | 17.78 ± 0.9 | 3.54 ± 1.0 | 5.66 ± 0 |
| IL-10 | 0.81 ± 0 | 4.84 ± 1.1 | 2.42 ± 0 | 3.23 ± 1.1 | 4.04 ± 0 |
| IFNγ• | 7.28 ± 0 | 4834.3 ± 0 | 84.16 ± 7.9 | 106.6 ± 34.4 | 134.7 ± 5.3 |

Vaginal washings were made the day 1 post-infection.
*Mediators and regulators of the inborn immunity
•Mediators and regulators of the adaptive immunity.
Note:
In order to avoid alterations in vaginal manipulation data, cytokines were tested at days 1 and 6 post-infection (p.i.). No major changes have been observed with respect to controls of day 6 p.i.

The invasion by an infective organism or foreign substances generates a complex but coordinated series of defense mechanisms in the host that includes inflammation along with the activation of the inborn and adaptive immunity activation. Cells from vascular endothelium are essential for immune and inflammatory processes. In response to a variety of stimulus, they carry out deep changes in the cytoskeleton structure, the expression and the activity of the cellular adhesion molecules, thrombotic and coagulating properties, and permeability of plasmatic proteins (Bevilacqua M P, 1993). Adhesion of leucocytes to the endothelium cells is particularly important as they finally determine the number, phenotype and function of the immune system cells that arrive to the inflammation site. Interaction regulation of the endothelial cells-leucocytes is mediated by a complex mechanism involving cytokines, adhesion surfaces and co-stimulating molecules (Kotowicz K et al., 2000).

Viral variants resistant (with a IC₅₀ of at least 4 times greater than the control IC₅₀) to aciclovir (ACV), as for example brivudin (BVDU) show mutations in the thymidine kinase (TK), while the viral variants that show resistance to drugs as foscarnet (PFA) (analogous to the pyrophosphate), PMEA and HPMPC (analogous to an acyclic nucleoside phosphonate), will suggest mutation in the gene of the viral DNA polymerase.

Thus, preliminary tests of the cloned variants of passages with greater selection pressure of 1C3 (17, 20 and 21) with pyrimidine and purine analogous as aciclovir (ACV) and brivudin (BVDU) would disclose alterations at viral TK level as it is shown in Table VIII, a characteristic that, up to date, has not been published in previous art documents. The TK gene is not essential for the in vitro viral replication, though in vivo, is involved in the virulence, pathogenicity and reactivation since latency (Andrei G et al., 2005). Though the sulphated polysaccharides act by blocking the viral enveloped glycoproteins, some studies show that the sulphated polysaccharides also act over a subsequent stage to the cellular bond in the viral cycle (Gonzalez M et al., 1987; Callahan L et al., 1991; De Vreesc K et al., 1996). Therefore, the selection pressure to which the viruses were subjected could have selected mutations in the TK gene and in additional superimposing genes generating strains with less in vivo pathogenic action (Jacobson J et al., 1993).

As the TK gene is superimposing the UL24 gene (locus syn, protein associated to a membrane) in the 5′ end, certain mutations in TK would also affect this gene as well as the other flanking UL22 coding the gH glycoprotein (Jacobson J et al., 1989). Therefore, as our viral variants have syn phenotype (synsytial), they would present modifications in the viral glycoproteins being thus possible to relate the phenotype and the genotype.

TABLE VIII
Susceptibility to viral variants

| | IC50^{a} (RR)^{b} | | | |
|---|---|---|---|---|
| Strains | Brivudin | Aciclovir | Foscarnet | Heparine |
| HSV-1 (F) | 0.01 | 0.05 | 6.12 | 1.25 |
| 1C3 syn | 0.02 (2.0) | 0.1 (2.0) | 3.12 (<1.0) | 7.1 (5.7) |
| 14-1 | | | | |
| HSV-1 TK⁻ | >0.16 (16.0) | >0.5 (10.0) | 35.1 (5.0) | 3.5 (3.0) |
| B2006 | | | | |
| 1C3 syn | 0.08 (8.0) | 0.28 (5.6) | 17.6 (2.9) | 10.0 (8.0) |
| 17-1 | | | | |
| 1C3 syn | 0.13 (13.0) | 0.2 (4.0) | 12.5 (2.0) | >20 (>16.0) |
| 17-2 | | | | |
| 1C3 syn | 0.08 (8.0) | 0.2 (4.0) | 17.6 (2.9) | 7.1 (5.7) |
| 17-3 | | | | |
| 1C3 syn | 0.08 (8.0) | 0.4 (8.0) | 12.5 (2.0) | >20 (>16.0) |
| 20-1 | | | | |
| 1C3 syn | 0.05 (5.6) | 0.2 (4.0) | 12.5 (2.0) | >20 (>16.0) |
| 20-2 | | | | |
| 1C3 syn | 0.06 (6.0) | 0.2 (4.0) | 17.6 (2.9) | >20 (>16.0) |
| 20-3 | | | | |
| 1C3 syn | 0.04 (4.0) | 0.2 (4.0) | 8.82 (1.4) | 10.0 (8.0) |
| 21-1 | | | | |
| 1C3 syn | 0.06 (6.0) | 0.4 (8.0) | 17.6 (2.9) | 10.0 (8.0) |
| 21-2 | | | | |
| 1C3 syn | 0.06 (6.0) | 0.2 (4.0) | 12.5 (2.0) | 7.1 (5.7) |
| 21-3 | | | | |

^{a}IC50 (μg/ml).
^{b}Relative resistance: relation between the IC50 of each clone and the IC50 of HSV-1 (F).

It is evident that 1C3 syn 14-1 does not show to have great alterations in the TK, due to the rare resistance to ACV and BVDU but even so it does not show pathogenic action with respect to the pattern strain in the intact mucosa. It is therefore supposed that variants with greater crossed resistance to drugs that have different mechanisms of action as the heparine and the BVDU or the ACV (as 1C3 syn 17-2, 1C3 syn 20-1) could be even more attenuated by other inoculation vias than 1C3 syn 14-1 and therefore there would exist more than one virulence expression site modified in this type of variants.

Neutralizing Antibodies

In order to determine a certain difference in the adaptive immune response for HSV-1 (F) y 1C3-syn-14-1, Balb/c mice were immunized with both strains. As it is shown in Table IX, serum from animals immunized with viral strains under study presented antibodies that neutralized, in the same way, the HSV-1 (F) and 1C3-syn-14-1 control strains, by using the direct plating methodology. Furthermore, immunized animals were challenged with the syncytial variant or the wild strain in a crossed way, without showing disease symptoms.

TABLE IX:
Titles of neutralizing antibodies immunized
with HSV-1 (F) and 1C3 syn 14-1

| | Title of 50% Ac. inh | Titles of 80% Ac. inh |
|---|---|---|
| Immunized with HSV-1 (F) | | |
| Syn 14-1 R1 | 100 | 64 |
| Syn 14-1 R2 | 209 | 112 |
| HSV-1 (F) R1 | 123 | 85 |
| HSV-1 (F) R2 | 320 | 100 |
| Immunized with Syn 14-1 | | |
| Syn 14-1 R1 | 160 | 68 |
| Syn 14-1 R2 | 80 | 50 |
| HSV-1 (F) R1 | 123 | 57 |
| HSV-1 (F) R2 | 58 | 40 |

The obtained results show that the sulphated polysaccharides are slow and poor inducers of viral resistance to drug (Witvrouw and De Clercq, 1997) due to the large process thereof for making the variants which generate and/or select stable phenotypical and genotypical modifications.

The present inventors propose the use of sulphated polymers (as for example the carrageenans) as selective agents and/or generators of attenuated viral mutants, showing in this case that the resistant to drugs HSV mutants that have been obtained, would have more than one modified determinant of virulence, as the viral TK gene and additional genes that would be responsible for the synsytial phenotype and of latency. As the TK gene is flanked by the UL22 gene (gH) and superimposed by the UL24 gene (containing a locus syn and codifying for a protein associated to a membrane), certainly, TK mutations would also be able to affect thereto (Jacobson J et al., 1993). Moreover gH along with gL, are essential viral proteins, that constitute a complex and would be involved in the entrance, outlet and spreading of the virus from cell-cell. This type of mutant would make possible to define the potential therapeutic or prophylactic use thereof as the viruses with these characteristics would be able to replicate with pathogenic decreased action (Coen D M et al., 1989) and cause latency without reactivation (Efstathiou S et al., 1989). Therefore, a way of accelerating the viral evolutive process could be set out, decreasing the virulence thereof, and consequently the pathogenicity thereof as it perpetuated into the host.

On the other hand, as during the experiments made by the present inventors, in the animals infected by nasal intact mucosa, normal births though premature occurred and that attenuated viral strains could be detected in the utero and in the vagina, without being possible to detect that these may be harmful for the breeding, it would be possible to set out that there exists a high possibility that vaccines containing attenuated viruses according to the inventive process could be used in pregnant animals and pregnant women. Furthermore, it is stated that if more than about 20 passages were made between the sulphated polymer and the virus susceptible to the inhibition of said polymer, with increasing concentrations of the sulphated polymer, starting from a sulphated polymer concentration in the first passage is less than the IC₅₀ of said virus amenable in wild state, there exists a great probability that the vaccines containing the attenuated strains according to the invention may be applied to pregnant animals and pregnant women.

### REFERENCES

1.—Andrei G, Snoeck R, De Clercq E. 1995. Antimicrob. Agents Chemother. 39, 1632-1635
2.—Andrei G, Balzarini J, Fiten P, De Clercq E, Opdenakker G, Snoeck R, 2005, J. Virol. 79:9.5863-5869.
3.—Basualdo J A, Coto C E, de Torres R A, 2006. Ed. Atlante. Microb. Biomemd. 79, 758-771.
4.—Beress A, Wassermann O, Bruñi T, Beress L, 1993. J. Nac. Prod. 56,478
5.—Bevilacqua M P, 1993, Annu. Rev. Immunol 11,767
6.—Bourgougnon N, Lahaye M, Chemann J C, Kornprobst J M, 1993 Bioorg. Med. Chem. Letters. 3: 1141-1146.
7.—Buck C B, Thompson C D, Roberts J N, Muller M, Lowy D R, Schiller J T, 2006. PlosPathogens 2:7, 671-680
8.—Cáceres P J, Carlucci M J, Damonte E B, Maatsuhiro B, Ziñiga EA, 2000, Phytochemistry. 53, 81.
9.—Callahan L, Pelan M, Mallinson M, Norcross M. A, 1991. J. Virol. 65: 1543-1550
10.—Carlucci M. J, Scolaro L. A, Damonte E. B, 1999a, Chemotherapy, 45:429
11.—Carlucci M J, Ciancia M, Matulewicz M C, Cerezo A. S, Damonte E. B, 1999b, Antiviral. Res. 43:93
12.—Carlucci M J, Pujol C A, Ciancia M, Noseda M, Matulewicz M C, Damonte E B, Cerezo A S, 1997, Int. J. Biol. Macromol. 20: -97-105.
13.—Carlucci M J, Scolaro L. A, Damonte E. B 2002, J. Med. Virol, 68:92
14.—Coen D M, Kosz-Vnenchak M, Jacobson J G, 1989, Proc. Natl. Acad. Sci. USA 86, 4736-4740
15.—Corey L, Wald A, Celum C. L, 2004, J. Acquired. Immun. Deficien. Syndrom. 35:435
16.—Damonte E B, Matulewicz M C, Cerezo A S, Coto C E, 1996, Chemoter. 42: 57-64.
17.—Damonte E B, Matulewicz M C, Cerezo A S, 2004. Current Med. Chem. 11, 2399-2419.
18.—De Vreesc K, Reymen D, Griffin P, Steinkasserer A, Werner G. J, Esté J, Henson G. W, Desmyter J, Anne J, De Clercq E, 1995. Antiviral Re. 29:209-219
19.—Duarte M E, Noseda D G, Noseda M E, Tulio S, Pujol C A, Damonte E B, 2001. Phytomedicine. 8,53
20.—Efstathiou S, Kemp S, Darby G, Minson A C, 1989, J. Gen. Virol. 70, 869-879
21.—Esko J D, Selleck S B, 2002. Annu. Rev. Biochem. 71: 435-471
22.—Evans J, Lock K, Levine B, Champness J, Sanderson M, Summers W, McLeish P, Buchan A, 1998, J. Gen. Virol, 79, 2083-2092
23.—Feldman S C, Reynaldi S, Stortz C A, Cerezo A S, Damonte E B. 1999. Phytomedicine, 6, 335
24.—Fleming D T, McQuillan G M, Johnson R E et al., N. Engl. J. Med, 1997, 337:1105
25.—Friedman H M, Wang L, Panqburn M. R, Lambris J. D, Lubinski J. 2000, J. Immunol. 15; 165(8):4528-36.
26.—Girond S, Crance J M, Van Cuyck-Gandre H, Renaudet J, Deloince R, 1991. Res. Virol 142:261-270
27.—Gonzalez M E, Alarcon B, Carrasco L, 1987. Antimicrob. Agents Chemother. 31 (9): 1388-1393
28.—Hashimoto K, Kodama E, Mori S, Watanabe J, Baba M, Okutani K, Matsuda M, Shigeta S. 1996. Antiviral Chem. Chemother. 7(4): 189-196.
29.—Haslin C, Lahaye M, Pellegrini M, Cheman J C, 2001, Planta Medica. 67, 301.
30.—Hasui M, Matsuda M, Okutani K, Shigeta S, 1995. Int. J. Biol. Macromol. 17, 293
31.—Hayashi K, Hamada J, Hayashi T, 1996. Phytother. Res. 10: 233-237.
32.—Hook E W, Cannon R O, Nahmias A J, et al., J. Infect. Dis 1992, 165:251
33.—Hoshino T, Hayashi T, Hayashi K, Hamada J, Lee J B, Sankawa U, 1998. Biol. Pharm. Bull. 21,730
34.—Huheihel M, Ishanu V, Tal J, Arad S M. 2002. J. Biochem. Biophys. Methods. 50,189
35.—Ihrcke N S, Parker W, Reissner K. J, Platt J. L. 1998, J. Cell. Physiol. 175:255-60
36.—Jacobson J, Martin S, Coen D, 1989, J. Virol. 63:1839-1843
37.—Jacobson J, Ruffner K, Kosz-vnenchak M, Hwang C, Wobbe K, Knipe D, Coen D, 1993, J. Virol, 67:11.6903-6908
38.—Johnston R. AIDS Patient Care STDs 2002, 16:419
39.—Kainulainen V H, Wang H, Schick C, Bernfield M, 1998, J. Biol. Chem. 273:11563-68
40.—Kawana T, Kawaguchi T, Sakamoto S, Lancet 1976, 7992:964
41.—Koelle D M, Wald A, 2000, J. Antimicr. Chemother, 45:1-8
42.—Kolender A, Pujol C A, Damonte E B, Matulewicz M C, Cerezo A S, 1997, Carbohydr. Res. 304: 53-60.
43.—Kotowicz K, Dixon G L, Klein Nj, Collard R E, 2000, Immunology 100:441
44.—Kurt-Jones E. A, Chan M, Zhou S, Wang J, Reed G, Bronson R, Arnold M. M, Knipe D. M, Finberg R. W. 2004, Proc. Natl. Acad. Sci. 101:(5)1315
45.—Larcher Cl, Gasser A, Hattmannstorfer R, Obexer P, Furhapter Ch, Frisch P, 2001, J. Invest. Dermat, 116:150-156
46.—Lee J B, Hayashi K, Hayashi T, Sankawa U, Maeda M. 1999. Planta Medica. 65, 439
47.—Leung M Y, Liu C, Koon J C, Fung K P, 2006, Immunology Letters 105,101
48.—Mazumder S, Ghosal P K, Pujol C A, Carlucci M J, Damonte E B, Ray B, 2002. Int. J. Biol. Macromol. 31,87
49.—Nakashima H, Kido Y, Kobayashi N, Neushul M, Yamamoto N, 1987. Antimicrob. Agent Chemoter. 31:1524-1528.
50.—Neumann J, Eis-Hubinger A. M, Koch N. 2003, J. Immunol 171:3075-83
51.—Novak N, Peng W M, 2005, Clin. Exp. Immunol, 142:405-410
52.—Oragui E, Nadel S, Kyd P, Levin M. 2000, Crit. Care. Med. 28:3002-08
53.—Painter T J, 1983. Academic Press, London, pp. 195-285
54.—Ponce N M A, Pujol C A, Damonte E B, Flores M I, Stortz C A, 2003. Carbohydr. Res. 338,153
55.—Pujol C. A, Carlucci M. J, Matulewicz M. C, Damonte E. B, 2007, Topics Heterocycl. Chem. Spring-Verlag
56.—Pujol C A, Errea M I, Matulewicz M C, Damonte E B, 1996, Phytother. Res. 10: 410-413.
57.—Pujol C A, Esteves J M, Carlucci M J, Ciancia M, Cerezo A S, Damonte E B, 2002. Antiviral Chem. Chemother. 13-83
58.—Pujol C A, Scolaro L. A, Ciancia M, Matulewicz M. C, Cerezo A. S, Damonte E. B, 2006, Plant. Med. 72:121
59.—Schacker T, Zeh J, Hu H. Shaughnessy M, Corey L. 2002, J. Infect. Diseases, 186, 1718-25
60.—Sekine H, Ohonuki N, Sadamasu K, Monma K, Nakamura H, Okada Y, Okuyama T, 1995. Chem. Pharm. Bull. 43(9): 1580-1584
61.—Snoeck R, Andrei G, De Clercq E, 1996. Eur. J. Clin. Microbiol. Infect. Dis. 15, 574-579
62.—Venkateswaran P S, Millman I, Blumberg B S, 1989. Planta Med. 55:265-270
63.—Wawer M J, Sewankambo N. K, Serwadda D, Quinn T C, Paxton L. A, Kiwanuka N, Wabwire-Mangen F, 1999, Project. Study. Group. Lancet 353:525-35
64.—Witvrouw and De Clercq, 1997. Gen. Pharmacol. 29:497-511
65.—Witvrouw M, Desmyter, De Clercq E, 1994. Antiviral Chem. Chemother. 5(6), 345-359.
66.—Witvrouw M, Este J A, Quinones Mateu M E, Andrei G, Snoeck R, Ikeda S, Pauwels R, Vittori Bianchini N, Desmyter J, De Clercq E, 1994, Antiviral Chem. Chemoter. 5: 297-303.

1. A process for preparing attenuated viral strains, characterized by comprising to contact at least one sulphated polymer and a virus susceptible to the inhibition of the polymer, through at least about 15 successive passages of the virus with increasing concentrations of the at least a sulphated polymer and where the concentration of the at least a sulphated polymer in the first passage is less than the IC₅₀ of the polymer for the amenable virus in wild state, as the amenable virus is selected by the method of reducing the viral plates and the attenuated virus strain has stable phenotypical and genotypical characteristics, different from the wild type virus strain that generated thereto.

2. The process for preparing attenuated viral strains according to claim 1, characterized by the at least one sulphated polymer is selected from the natural sulphated polymers and the synthetic sulphated polymers.

3. The process for preparing attenuated viral strains according to claim 1, characterized by the at least one sulphated polymer is a sulphated polysaccharide.

4. The process for preparing attenuated viral strains according to claim 1, characterized by the at least one sulphated polysaccharide is a carrageenan.

5. The process for preparing attenuated viral strains according to claim 2, characterized by the at least one synthetic sulphated polymer is a synthetic naphthalene sulphated polymer.

6. The process for preparing attenuated viral strains according to claim 1, characterized by the at least one sulphated polymer is a glycosaminoglican or a sulphated polymer with similar structure to a glycosaminoglican.

7. The process for preparing attenuated viral strains according to claim 1, characterized by the amenable virus is the Herpes virus.

8. The process for preparing attenuated viral strains according to claim 1, characterized by the Herpes virus is the Herpes simplex virus type 1.

9. The process for preparing attenuated viral strains according to claim 5, characterized by the herpes virus is the Herpes simplex virus type 2.

10. The process for preparing attenuated viral strains according to claim 1, characterized by contacting one sulphated polymer and a virus susceptible to the inhibition of the polymer.

11. The process for preparing attenuated viral strains according to claim 1, characterized by the sulphated polymer is a sulphated polysaccharide.

12. The process for preparing attenuated viral strains according to claim 1, characterized by the sulphated polysaccharide is a carrageenan.

13. The process for preparing attenuated viral strains according to claim 10, characterized by the so obtained attenuated viral strain has the viral structure thereof altered.

14. The process for preparing attenuated viral strains according to claim 10, characterized by the so obtained attenuated viral strain has the envelope thereof altered.

15. An attenuated virus characterized by being obtained by the process of claim 1, which virus has modified the viral structure thereof, as it is resistant to the sulphated polysaccharides of a structure similar to that used in the process, and has an IC₅₀ that is about 4 times higher than the IC₅₀ of the wild virus.

16. Use of an attenuated virus strain by the process of claim 1, characterized by being used in the preparation of vaccines.

17. Use of an attenuated virus strain by the process of claim 1, characterized by being used in the preparation of pharmaceutical compositions with therapeutic or prophylactic activity.

18. A vaccine based on live attenuated viruses characterized by comprising attenuated viruses obtained according to the process in accordance with claim 1.

19. A vaccine based on live attenuated viruses characterized by comprising one or more types of attenuated viruses obtained according to a process in accordance with claim 1.
